(19) United States
(12) Patent Application Publication
(10) Pub. No.: US 2015/0362473 A1
(43) Pub. Date: Dec. 17, 2015

(54) Low-E Panels Utilizing High-Entropy Alloys and Combinatorial Methods and Systems for Developing the Same

(71) Applicant: Intermolecular Inc., San Jose, CA (US)

(72) Inventor: Abraham Anapolsky, San Mateo, CA (US)

(21) Appl. No.: 14/303,277

(22) Filed: Jun. 12, 2014

(51) Int. Cl. G01N 33/20

(52) U.S. Cl. CPC G01N 33/20

(57) ABSTRACT

Embodiments provided herein describe low-e panels utilizing high-entropy alloys (HEAs) and methods for forming such low-e panels, as well as combinatorial methods and systems for developing such low-e panels. A transparent substrate is provided. A reflective layer is formed above the transparent substrate. A metallic layer is formed above the transparent substrate. The metallic layer includes an HEA. The metallic layer, or any other component of the low-panels, may be formed using combinatorial processing.

The present invention relates to low-e panels. More particularly, this invention relates to low-e panels utilizing high-entropy alloys (HEAs) and combinatorial methods and systems for developing such low-e panels.

### BACKGROUND OF THE INVENTION

Low emissivity, or low-e, panels are often formed by depositing a reflective layer (e.g., silver), along with various other layers, onto a transparent (e.g., glass) substrate. The other layers typically include various dielectric and metal oxide layers, such as silicon nitride, tin oxide, and zinc oxide, to provide a barrier between the stack and both the substrate and the environment, as well as to act as optical fillers and improve the optical characteristics of the panel.

It is also desirable for the stack of layers (or the low-e panel as a whole) provide the same optical and thermal performance before and after undergoing a heat treatment (e.g., to temper the glass). Many conventional metal films cannot satisfy these requirements because at the required thickness, the metal films are subject to mechanical damage and environmental corrosion due to the fine grain structure. In addition, deposited metal films are subjected to stress both before and after the heat treatment, resulting in undesirable effects on the metal films and the glass substrate.

For conventional low-e films stacks utilizing silver in the reflective layer, a metallic barrier film is typically incorporated between the silver and subsequent dielectric layers to prevent diffusion of, for example, oxygen into the silver. In order to provide a sufficient barrier for the silver, the barrier layers are usually partially oxidized. Even with the most current barrier solutions for silver, usually silver-based low-e panels must be assembled into glazing units (e.g., double-pane window) within a limited time (e.g., hours) of heat treatment of the glass. Otherwise, the panels may be rendered esthetically and/or functionally useless.

### BRIEF DESCRIPTION OF THE DRAWINGS

To facilitate understanding, identical reference numerals have been used, where possible, to designate identical elements that are common to the figures. The drawings are not to scale and the relative dimensions of various elements in the drawings are depicted schematically and not necessarily to scale.

The techniques of the present invention can readily be understood by considering the following detailed description in conjunction with the accompanying drawings, in which:

FIG. 1 illustrates a schematic diagram for implementing combinatorial processing and evaluation using primary, secondary, and tertiary screening.

FIG. 2 is a simplified schematic diagram illustrating a general methodology for combinatorial process sequence integration that includes site isolated processing and/or conventional processing in accordance with some embodiments.

FIG. 3 is a simplified schematic diagram illustrating an integrated high productivity combinatorial (HPC) system in accordance with some embodiments.

FIG. 4 is a simplified schematic diagram illustrating a sputter processing chamber configured to perform combinatorial processing and full substrate processing in accordance with some embodiments.

FIG. 5 is a simplified schematic diagram illustrating a sputter processing gun configured to perform combinatorial processing and full substrate processing in accordance with some embodiments.

FIG. 6 is a cross-sectional view of a low-e panel according to some embodiments.

FIG. 7 is a flow chart illustrating a method for forming low-e panels according to some embodiments.

### DETAILED DESCRIPTION

A detailed description of one or more embodiments is provided below along with accompanying figures. The detailed description is provided in connection with such embodiments, but is not limited to any particular example. The scope is limited only by the claims, and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For the purpose of clarity, technical material that is known in the technical fields related to the embodiments has not been described in detail to avoid unnecessarily obscuring the description.

The term “horizontal” as used herein will be understood to be defined as a plane parallel to the plane or surface of the substrate, regardless of the orientation of the substrate. The term “vertical” will refer to a direction perpendicular to the horizontal as previously defined. Terms such as “above”, “below”, “bottom”, “top”, “side” (e.g. sidewall), “higher”, “lower”, “upper”, “over”, and “under”, are defined with respect to the horizontal plane. The term “on” means there is direct contact between the elements. The term “above” will allow for intervening elements.

In some embodiments, high-entropy alloys (HEAs) are used to form low-e panels. HEAs generally consist of five or more metallic elements in appreciable concentrations (e.g., 5-35% by weight of each element) which form in an amorphous state due to the inability to form a crystal lattice, perhaps due to divergent atomic diameters. HEAs have at least two desirable qualities due to their amorphous nature: high hardness due to the lack of dislocations and immobility of the matrix atoms, and high resistivity to corrosion due to the lack of grain boundaries.

Exemplary elements that may be used in the HEAs described herein include iron, zinc, zirconium, aluminum, titanium, tungsten, tantalum, hafnium, copper, boron, niobium, chromium, hafnium, and/or any combination thereof. The HEA(s) may be deposited as a layer (e.g., via physical vapor deposition (PVD)) in a low-e stack above a transparent substrate, such as glass. Other layers that may also be included in the low-e stack include, for example, a reflective layer and at least one dielectric layer (e.g., silicon nitride).

In some embodiments, combinatorial methods and systems for evaluating and developing the use of HEAs in low-e panels are also provided. In some embodiments, a plurality of regions (e.g., site-isolated regions) are designated on at least one substrate (e.g., a glass substrate). A first HEA material is formed on a first of the plurality of regions on the at least one substrate with a first set of processing conditions. A second HEA material is formed on a second of the plurality of regions on the at least one substrate with a second set of processing conditions. The second set of processing conditions is different than the first set of processing conditions. However, it should be understood, that in some embodiments, the use of the same set of processing conditions may be repeated on several of the regions (or one or more substrate) to test for consistency and repeatability.

The first HEA material and the second HEA material may then be characterized. In some embodiments, the characterizing of the HEA material(s) includes testing or evaluating the HEA material(s) with respect to properties relevant to the use of the HEA material(s) in low-e panels (e.g., transmittance, reflectance, color, emissivity, thickness, durability, barrier performance, etc.). One of the first set of processing conditions and the second set of processing conditions may be selected based on the characterizing of the first HEA material and the second HEA material.

As such, in accordance with some embodiments, combinatorial processing may be used to produce and evaluate different materials, substrates, chemicals, consumables, processes, coating stacks, and techniques related to HEA materials, as well as other materials/layers used in low-e panels, as well as build structures or determine how HEA materials coat, fill or interact with existing structures in order to vary materials, unit processes and/or process sequences across multiple site-isolated regions on the substrate(s). These variations may relate to specifications such as temperatures, exposure times, layer thicknesses, chemical compositions of majority and minority elements of layers, gas compositions, chemical compositions of wet and dry surface chemistries, power and pressure of sputter deposition conditions, humidity, etc. of the formulations and/or the substrates at various stages of the screening processes described herein. However, it should be noted that in some embodiments, the chemical composition (e.g., of the HEA material and/or of the other components) remains the same, while other parameters are varied, and in other embodiments, the chemical composition is varied.

As part of the discovery, optimization and qualification of each unit process, it is desirable to be able to i) test different materials, ii) test different processing conditions within each unit process module, iii) test different sequencing and integration of processing modules within an integrated processing tool, iv) test different sequencing of processing tools in executing different process sequence integration flows, and combinations thereof in the manufacture of devices such as integrated circuits. In particular, there is a need to be able to test i) more than one material, ii) more than one processing condition, iii) more than one sequence of processing conditions, iv) more than one process sequence integration flow, and combinations thereof, collectively known as “combinatorial process sequence integration,” on a single monolithic substrate without the need of consuming the equivalent number of monolithic substrates per material(s), processing condition(s), sequence(s) of processing conditions, sequence(s) of processes, and combinations thereof. This can greatly improve both the speed and reduce the costs associated with the discovery, implementation, optimization, and qualification of material(s), process(es), and process integration sequence(s) required for manufacturing.

Systems and methods for High Productivity Combinatorial (HPC) processing are described in U.S. Pat. No. 7,544,574, filed on Feb. 10, 2006, U.S. Pat. No. 7,824,935, filed on Jul. 2, 2008, U.S. Pat. No. 7,871,928, filed on May 4, 2009, U.S. Pat. No. 7,902,063, filed on Feb. 10, 2006, and U.S. Pat. No. 7,947,531, filed on Aug. 28, 2009, which are all herein incorporated by reference. Systems and methods for HPC processing are further described in U.S. patent application Ser. No. 11/352,077, filed on Feb. 10, 2006, claiming priority from Oct. 15, 2005, U.S. patent application Ser. No. 11/419,174, filed on May 18, 2006, claiming priority from Oct. 15, 2005, U.S. patent application Ser. No. 11/674,132, filed on Feb. 12, 2007, claiming priority from Oct. 15, 2005, and U.S. patent application Ser. No. 11/674,137, filed on Feb. 12, 2007, claiming priority from Oct. 15, 2005 which are all herein incorporated by reference.

HPC processing techniques have been successfully adapted to wet chemical processing such as etching and cleaning HPC processing techniques have also been successfully adapted to deposition processes such as physical vapor deposition (PVD), atomic layer deposition (ALD), and chemical vapor deposition (CVD).

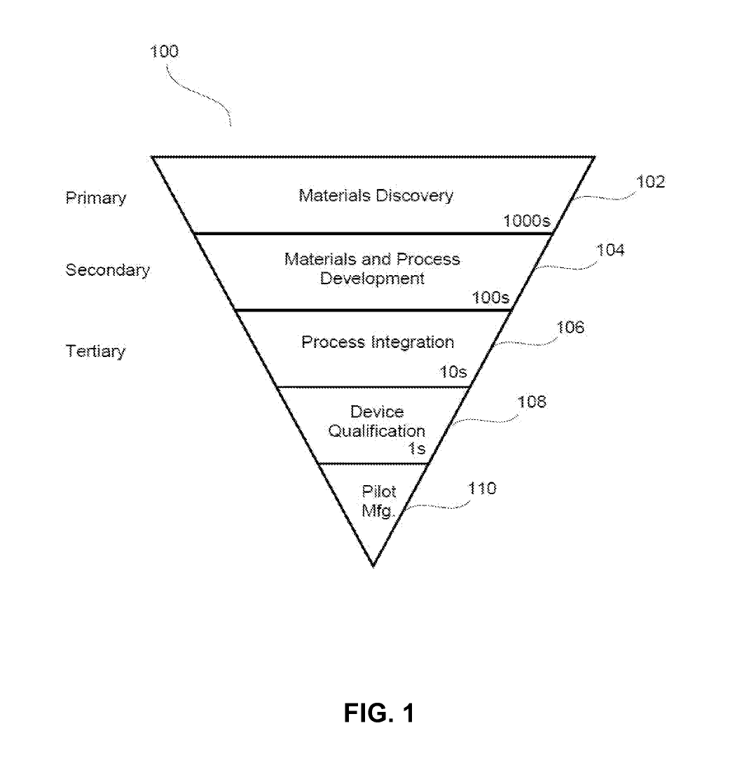

FIG. 1 illustrates a schematic diagram 100 for implementing combinatorial processing and evaluation using primary, secondary, and tertiary screening. The schematic diagram 100 illustrates that the relative number of combinatorial processes run with a group of substrates decreases as certain materials and/or processes are selected. Generally, combinatorial processing includes performing a large number of processes during a primary screen, selecting promising candidates from those processes, performing the selected processing during a secondary screen, selecting promising candidates from the secondary screen for a tertiary screen, and so on. In addition, feedback from later stages to earlier stages can be used to refine the success criteria and provide better screening results.

For example, thousands of materials are evaluated during a materials discovery stage 102. Materials discovery stage 102 is also known as a primary screening stage performed using primary screening techniques. Primary screening techniques may include dividing substrates into coupons and depositing materials using varied processes. The materials are then evaluated (e.g., with respect to properties relevant to use of HEA material(s) in low-e panels), and promising candidates are advanced to the secondary screen, or materials and process development stage 104. Evaluation of the materials is performed using metrology tools such as ellipsometers, XRF, stylus profilers, hall measurements, optical transmission, reflection, and absorption testers, electronic testers and imaging tools (i.e., microscopes).

The materials and process development stage 104 may evaluate hundreds of materials (i.e., a magnitude smaller than the primary stage) and may focus on the processes used to deposit or develop those materials. Promising materials and processes are again selected, and advanced to the tertiary screen or process integration stage 106, where tens of materials and/or processes and combinations are evaluated. The tertiary screen or process integration stage 106 may focus on integrating the selected processes and materials with other processes and materials.

The most promising materials and processes from the tertiary screen are advanced to device qualification 108. In device qualification, the materials and processes selected are evaluated for high volume manufacturing, which normally is conducted on full substrates within production tools, but need not be conducted in such a manner. The results are evaluated to determine the efficacy of the selected materials and processes. If successful, the use of the screened materials and processes can proceed to pilot manufacturing 110.

The schematic diagram 100 is an example of various techniques that may be used to evaluate and select materials and processes for the development of new materials and processes. The descriptions of primary, secondary, etc. screening and the various stages 102-110 are arbitrary and the stages may overlap, occur out of sequence, be described and be performed in many other ways.

This application benefits from High Productivity Combinatorial (HPC) techniques described in U.S. patent application Ser. No. 11/674,137, filed on Feb. 12, 2007, which is hereby incorporated for reference in its entirety. Portions of the '137 application have been reproduced below to enhance the understanding of the present invention. The embodiments described herein enable the application of combinatorial techniques to process sequence integration in order to arrive at a globally optimal sequence of, for example, device manufacturing operations by considering interaction effects between the unit manufacturing operations, the process conditions used to effect such unit manufacturing operations, hardware details used during the processing, as well as materials characteristics of components utilized within the unit manufacturing operations. Rather than only considering a series of local optimums (i.e., where the best conditions and materials for each manufacturing unit operation is considered in isolation), the embodiments described below consider interactions effects introduced due to the multitude of processing operations that are performed and the order in which such multitude of processing operations are performed when fabricating a device. A global optimum sequence order is therefore derived and as part of this derivation, the unit processes, unit process parameters and materials used in the unit process operations of the optimum sequence order are also considered.

The embodiments described further analyze a portion or sub-set of the overall process sequence used to manufacture a device. Once the subset of the process sequence is identified for analysis, combinatorial process sequence integration testing is performed to optimize the materials, unit processes, hardware details, and process sequence used to build that portion of the device or structure. During the processing of some embodiments described herein, structures are formed on the processed substrate that are equivalent to the structures formed during actual production of the device. For example, such structures may include, but would not be limited to, barrier layers, reflective layers, dielectric layers, or any other series of layers or unit processes that create an intermediate structure found on devices such as low-e panels. While the combinatorial processing varies certain materials, unit processes, hardware details, or process sequences, the composition or thickness of the layers or structures or the action of the unit process, such as cleaning, surface preparation, deposition, surface treatment, etc. is substantially uniform through each discrete region. Furthermore, while different materials or unit processes may be used for corresponding layers or steps in the formation of a structure in different regions of the substrate during the combinatorial processing, the application of each layer or use of a given unit process is substantially consistent or uniform throughout the different regions in which it is intentionally applied. Thus, the processing is uniform within a region (inter-region uniformity) and between regions (intra-region uniformity), as desired. It should be noted that the process can be varied between regions, for example, where a chemical composition or thickness of a layer is between the regions, etc., as desired by the design of the experiment.

The result is a series of regions on the substrate that contain structures or unit process sequences that have been uniformly applied within that region and, as applicable, across different regions. This process uniformity allows comparison of the properties within and across the different regions such that the variations in test results are due to the varied parameter (e.g., materials, unit processes, unit process parameters, hardware details, or process sequences) and not the lack of process uniformity. In the embodiments described herein, the positions of the discrete regions on the substrate can be defined as needed, but are preferably systematized for ease of tooling and design of experimentation. In addition, the number, variants and location of structures within each region are designed to enable valid statistical analysis of the test results within each region and across regions to be performed.

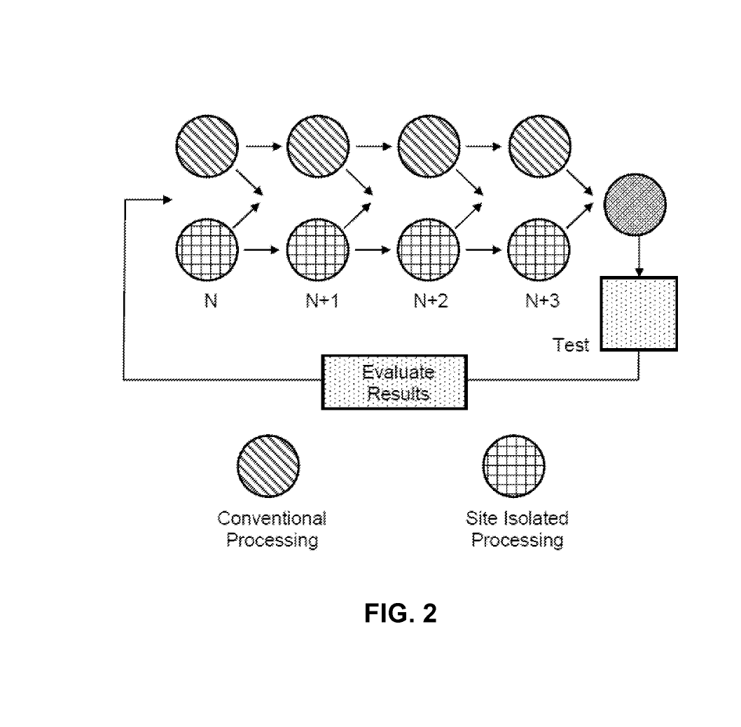

FIG. 2 is a simplified schematic diagram illustrating a general methodology for combinatorial process sequence integration that includes site isolated processing and/or conventional processing in accordance with some embodiments. In some embodiments, the substrate is initially processed using conventional process N. In some embodiments, the substrate is then processed using site isolated process N+1. During site isolated processing, an HPC module may be used, such as the HPC module described in U.S. patent application Ser. No. 11/352,077, filed on Feb. 10, 2006. The substrate can then be processed using site isolated process N+2, and thereafter processed using conventional process N+3. Testing is performed and the results are evaluated. The testing can include physical, chemical, acoustic, magnetic, electrical, optical, etc. tests. From this evaluation, a particular process from the various site isolated processes (e.g. from steps N+1 and N+2) may be selected and fixed so that additional combinatorial process sequence integration may be performed using site isolated processing for either process N or N+3. For example, a next process sequence can include processing the substrate using site isolated process N, conventional processing for processes N+1, N+2, and N+3, with testing performed thereafter.

It should be appreciated that various other combinations of conventional and combinatorial processes can be included in the processing sequence with regard to FIG. 2. That is, the combinatorial process sequence integration can be applied to any desired segments and/or portions of an overall process flow. Characterization, including physical, chemical, acoustic, magnetic, electrical, optical, etc. testing, can be performed after each process operation, and/or series of process operations within the process flow as desired. The characterization (or testing) may be performed using various methods, such as ellipsometry, atomic force microscopy (AFM), scanning electron microscopy (SEM), optical transmission and reflectance testing, X-Ray Diffraction (XRD), X-Ray Fluorescence (XRF), Fourier Transform Infrared (FTIR) spectroscopy, or any combination thereof.

The feedback provided by the testing is used to select certain materials, processes, process conditions, and process sequences and eliminate others. Furthermore, the above flows can be applied to entire monolithic substrates, or portions of monolithic substrates such as coupons.

Under combinatorial processing operations the processing conditions at different regions can be controlled independently. Consequently, process material amounts, reactant species, processing temperatures, processing times, processing pressures, processing flow rates, processing powers, processing reagent compositions, the rates at which the reactions are quenched, deposition order of process materials, process sequence steps, hardware details, etc., can be varied from region to region on the substrate. Thus, for example, when exploring materials, a processing material delivered to a first and second region can be the same or different. If the processing material delivered to the first region is the same as the processing material delivered to the second region, this processing material can be offered to the first and second regions on the substrate at different concentrations. In addition, the material can be deposited under different processing parameters. Parameters which can be varied include, but are not limited to, process material amounts, reactant species, processing temperatures, processing times, processing pressures, processing flow rates, processing powers, processing reagent compositions, the rates at which the reactions are quenched, atmospheres in which the processes are conducted, an order in which materials are deposited, hardware details of the gas distribution assembly, etc. It should be appreciated that these process parameters are exemplary and not meant to be an exhaustive list as other process parameters commonly used in device manufacturing may be varied.

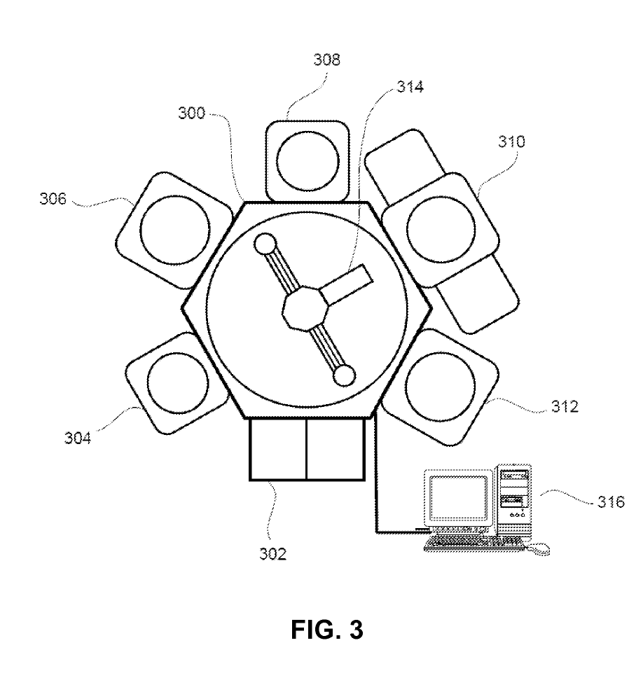

FIG. 3 is a simplified schematic diagram illustrating an integrated high productivity combinatorial (HPC) system in accordance with some embodiments. HPC system includes a frame 300 supporting a plurality of processing modules. It should be appreciated that frame 300 may be a unitary frame in accordance with some embodiments. In some embodiments, the environment within frame 300 is controlled. Load lock/factory interface 302 provides access into the plurality of modules of the HPC system. Robot 314 provides for the movement of substrates (and masks) between the modules and for the movement into and out of the load lock 302. Modules (or processing tools) 304-312 may be any set of modules and preferably include one or more combinatorial modules. For example, module 304 may be an orientation/degassing module, module 306 may be a clean module, either plasma or non-plasma based, modules 308 and/or 310 may be combinatorial/conventional dual purpose modules. Module 312 may provide conventional clean or degas as necessary for the experiment design.

Any type of chamber or combination of chambers may be implemented and the description herein is merely illustrative of one possible combination and not meant to limit the potential chamber or processes that can be supported to combine combinatorial processing or combinatorial plus conventional processing of a substrate or wafer. In some embodiments, a centralized controller, i.e., computing device 316, may control the processes of the HPC system, including the power supplies and synchronization of the duty cycles described in more detail below. Further details of one possible HPC system are described in U.S. application Ser. No. 11/672,478 filed Feb. 7, 2007, now U.S. Pat. No. 7,867,904 and claiming priority to U.S. Provisional Application No. 60/832,248 filed on Jul. 19, 2006, and U.S. application Ser. No. 11/672,473, filed Feb. 7, 2007, and claiming priority to U.S. Provisional Application No. 60/832,248 filed on Jul. 19, 2006, which are all herein incorporated by reference. With HPC system, a plurality of methods may be employed to deposit material upon a substrate employing combinatorial processes.

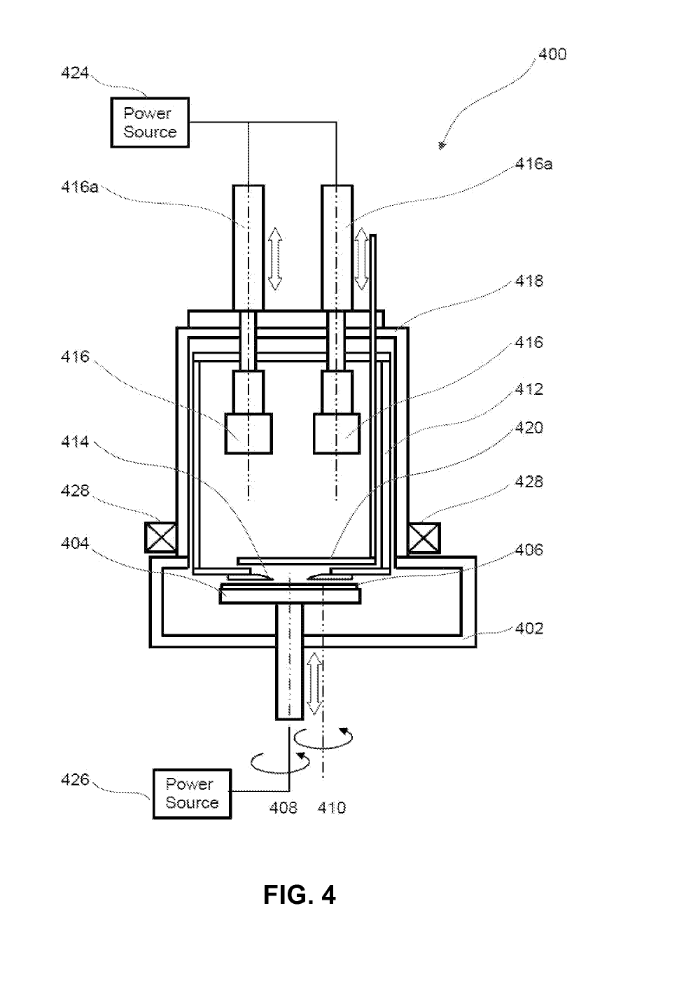

FIG. 4 is a simplified schematic diagram illustrating a PVD chamber (or processing tool), more particularly, a sputter chamber, configured to perform combinatorial processing and full substrate processing in accordance with some embodiments. Processing chamber 400 includes a bottom chamber portion 402 disposed under top chamber portion 418. Within bottom portion 402, substrate support 404 is configured to hold a substrate 406 disposed thereon and can be any known substrate support, including but not limited to a vacuum chuck, electrostatic chuck or other known mechanisms. Substrate support 404 is capable of both rotating around its own central axis 408 (referred to as “rotation” axis), and rotating around an exterior axis 410 (referred to as “revolution” axis). Such dual rotary substrate support is central to combinatorial processing using site-isolated mechanisms. Other substrate supports, such as an XY table, can also be used for site-isolated deposition. In addition, substrate support 404 may move in a vertical direction. It should be appreciated that the rotation and movement in the vertical direction may be achieved through known drive mechanisms which include magnetic drives, linear drives, worm screws, lead screws, a differentially pumped rotary feed through drive, etc. Power source 426 provides a bias power to substrate support 404 and substrate 406 and produces a negative bias voltage on substrate 406. In some embodiments, power source 426 provides a radio frequency (RF) power sufficient to take advantage of the high metal ionization to improve step coverage of vias and trenches of patterned wafers. In some embodiments, the RF power supplied by power source 426 is pulsed and synchronized with the pulsed power from power source 424.

Substrate 406 may be a conventional round 200 mm, 300 mm, or any other larger or smaller substrate/wafer size. In some embodiments, substrate 406 may be a square, rectangular, or other shaped substrate. In some embodiments, substrate 406 is made of glass. However, in other embodiments, the substrate 406 is made of a semiconductor material, such as silicon. One skilled in the art will appreciate that substrate 406 may be a blanket substrate, a coupon (e.g., partial wafer), or even a patterned substrate having predefined regions. In some embodiments, substrate 406 may have regions defined through the processing described herein. The term region is used herein to refer to a localized (or site-isolated) area on a substrate which is, was, or is intended to be used for processing or formation of a selected material. The region can include one region and/or a series of regular or periodic regions predefined on the substrate. The region may have any convenient shape, e.g., circular, rectangular, elliptical, wedge-shaped, etc. In the semiconductor field, a region may be, for example, a test structure, single die, multiple dies, portion of a die, other defined portion of substrate, or an undefined area of a substrate, e.g., blanket substrate which is defined through the processing.

Top chamber portion 418 of chamber 400 in FIG. 4 includes process kit shield 412, which defines a confinement region over a radial portion of substrate 406. Process kit shield 412 is a sleeve having a base (optionally integrated with the shield) and an optional top within chamber 400 that may be used to confine a plasma generated therein. The generated plasma will dislodge atoms from a target and the sputtered atoms will deposit on an exposed surface of substrate 406 to combinatorial process regions of the substrate in a site-isolated manner (e.g., such that only the particular region on the substrate is processed) in some embodiments. In other embodiments, full wafer processing can be achieved by optimizing gun tilt angle and target-to-substrate spacing, and by using multiple process guns 416. Process kit shield 412 is capable of being moved in and out of chamber 400 (i.e., the process kit shield is a replaceable insert). In other embodiments, process kit shield 412 remains in the chamber for both the full substrate and combinatorial processing. Process kit shield 412 includes an optional top portion, sidewalls and a base. In some embodiments, process kit shield 412 is configured in a cylindrical shape, however, the process kit shield may be any suitable shape and is not limited to a cylindrical shape.

The base of process kit shield 412 includes an aperture 414 through which a surface of substrate 406 is exposed for deposition or some other suitable semiconductor processing operations. Aperture shutter 420 which is moveably disposed over the base of process kit shield 412. Aperture shutter 420 may slide across a bottom surface of the base of process kit shield 412 in order to cover or expose aperture, 414, in some embodiments. In other embodiments, aperture shutter 420 is controlled through an arm extension which moves the aperture shutter to expose or cover aperture 414. It should be noted that although a single aperture is illustrated, multiple apertures may be included. Each aperture may be associated with a dedicated aperture shutter or an aperture shutter can be configured to cover more than one aperture simultaneously or separately. Alternatively, aperture 414 may be a larger opening and aperture shutter 420 may extend with that opening to either completely cover the aperture or place one or more fixed apertures within that opening for processing the defined regions. The dual rotary substrate support 404 is central to the site-isolated mechanism, and allows any location of the substrate or wafer to be placed under the aperture 414. Hence, the site-isolated deposition is possible at any location on the wafer/substrate.

Although only two process guns 416 are visible in FIG. 4, any number of process guns may be included (e.g., one, three, four or more process guns). Process guns 416 are moveable in a vertical direction so that one or both of the guns may be lifted from the slots of the shield. Where more than one process gun is included, the plurality of process guns may be referred to as a cluster of process guns. In some embodiments, process guns 416 are oriented or angled so that a normal reference line extending from a planar surface of the target of the process gun is directed toward an outer periphery of the substrate in order to achieve good uniformity for full substrate deposition. The target/gun tilt angle depends on the target size, target-to-substrate spacing, target material, process power/pressure, etc.

Top chamber portion 418 of chamber 400 of FIG. 4 includes sidewalls and a top plate which house process kit shield 412. Arm extensions 416a which are fixed to process guns 416 may be attached to a suitable drive, (i.e., lead screw, worm gear, etc.), configured to vertically move process guns 416 toward or away from a top plate of top chamber portion 418. Arm extensions 416a may be pivotally affixed to process guns 416 to enable the process guns to tilt relative to a vertical axis. In some embodiments, process guns 416 tilt toward aperture 414 when performing combinatorial processing and tilt toward a periphery of the substrate being processed when performing full substrate processing. It should be appreciated that process guns 416 may tilt away from aperture 414 when performing combinatorial processing in other embodiments. In yet other embodiments, arm extensions 416a are attached to a bellows that allows for the vertical movement and tilting of process guns 416. Arm extensions 416a enable movement with four degrees of freedom in some embodiments. Where process kit shield 412 is utilized, the aperture openings are configured to accommodate the tilting of the process guns. The amount of tilting of the process guns may be dependent on the process being performed in some embodiments.

Power source 424 provides power for sputter guns 416 whereas power source 426 provides RF bias power to an electrostatic chuck. As mentioned above, the output of power source 426 is synchronized with the output of power source 424. It should be appreciated that power source 424 may output a direct current (DC) power supply or a radio frequency (RF) power supply. In other embodiments, the DC power is pulsed and the duty cycle is less than 30% on-time at maximum power in order to achieve a peak power of 10-15 kilowatts. Thus, the peak power for high metal ionization and high density plasma is achieved at a relatively low average power which will not cause any target overheating/cracking issues. It should be appreciated that the duty cycle and peak power levels are exemplary and not meant to be limiting as other ranges are possible and may be dependent on the material and/or process being performed.

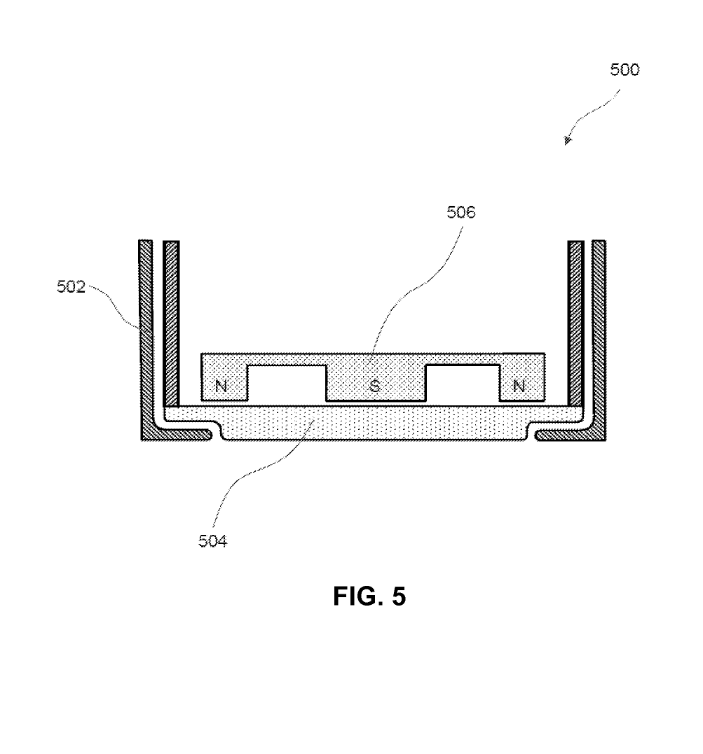

FIG. 5 illustrates a portion of a sputter gun 500 that would be part of the sputter guns 416 in FIG. 4. Illustrated in FIG. 5 is a grounded shield 502 surrounding the exterior of the target 504 and magnetron 506 assembly. As will be appreciated by one skilled in the art, the target 504 (or the target 504 of each of the provided sputter guns 500) includes (or is made of) a material(s) to be deposited on the substrate 406. In some embodiments, the various materials included in the target(s) are suitable for forming HEAs. Exemplary materials include metallic elements such as iron, zinc, zirconium, aluminum, titanium, tungsten, tantalum, hathium, copper, boron, niobium, chromium, hafnium, and combinations thereof. In some embodiments, the target(s) 504, when considered in combination, include at least five (i.e., five or more) metallic elements. For example, when a total of four sputter guns (and four targets) are used, three of the targets may each include a single element while the fourth target includes an alloy of two or three metallic elements (i.e., thus providing an HEA material made of five or six elements).

Using processing chamber 400, perhaps in combination with other processing tools, HEA materials may be developed and evaluated in the manner described above. In particular, in some embodiments, HEA materials may be formed (or deposited) on different (i.e., two or more) site-isolated regions of substrate 406 (or on multiple substrates) under varying processing conditions (including, for example, the formation/deposition of different HEA materials). For example, (a first) HEA material may be ejected from one of more of targets 504 and deposited onto a first of the regions on substrate 406 under a first set of processing conditions, and either sequentially or simultaneously, (a second) HEA material may be ejected from one of more of targets 504 and deposited onto a second of the regions on substrate 406 under a different, second set of processing conditions.

The HEA material(s) (and/or processing conditions) may then be characterized. In some embodiments, the characterizing of the HEA material(s) includes testing or evaluating the HEA material(s) with respect to properties relevant to the use of the HEA material(s) in low-e panels (e.g., transmittance, reflectance, color, emissivity, thickness, durability, barrier performance, etc.). Particular materials and/or processing conditions may then be selected (e.g., for further testing or use in devices) based on the desired parameters or properties.

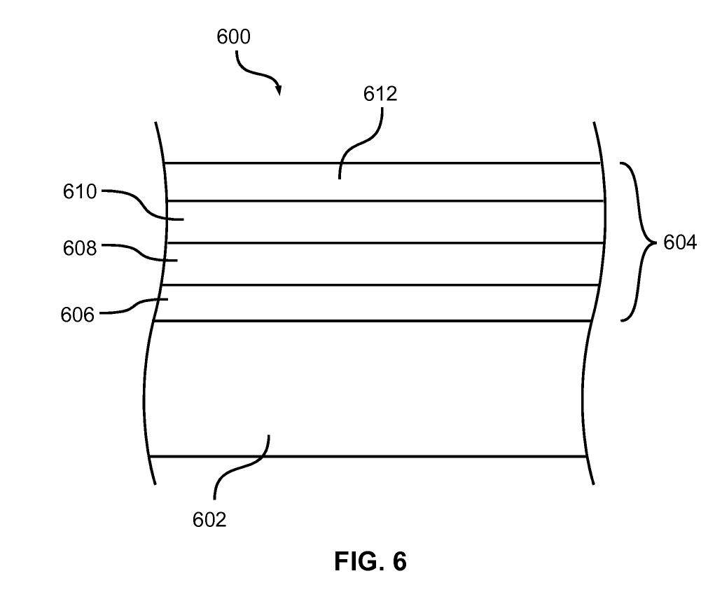

It should be understood that the development of the HEA materials may involve the use of multiple processing tools, such as modules 304-312 in FIG. 3. For example, various other materials/layers (e.g., as shown in FIG. 6), in addition to the HEA material, may be formed on each site-isolated region on the substrate, and additional processing steps, such as cleanings, may be performed at various stages of the processing, in processing tools/chambers different from the one in which the HEA material(s) is formed. This processing may utilize several of the modules 304-312 and involve transporting the substrate between the modules in a controlled environment (e.g., without breaking vacuum).

FIG. 6 illustrates an exemplary low-e panel 600 according to some embodiments. The low-e panel 600 includes a transparent substrate 602 and a low-e stack 604 formed above the transparent substrate 602. The transparent substrate 602 in some embodiments is made of a low-emissivity glass, such as borosilicate glass. However, in some embodiments, the transparent substrate 602 may be made of plastic or a transparent polymer, such as polyethylene terephthalate (PET), poly(methyl methacrylate) (PMMA), polycarbonate (PC), and polyimide (PI). The transparent substrate 602 has a thickness of, for example, between about 1 and about 10 millimeters (mm). In a testing environment, the transparent substrate 602 may be round with a diameter of, for example, about 200 or about 300 mm. However, in a manufacturing environment, the transparent substrate 602 may be square or rectangular and significantly larger (e.g., about 0.5-about 4 meters (m) across).

The low-e stack 604 includes a protective layer 606, a reflective layer 608, an HEA layer 610, and a capping layer 612. Exemplary details as to the functionality provided by each of the layers 106-126 are provided below.

The various layers in the low-e stack 604 may be formed sequentially (i.e., from bottom to top) above the transparent substrate 102 using, for example, a physical vapor deposition (PVD) and/or reactive sputtering processing tool. In some embodiments, the low-e stack 604 is formed above the entire substrate 602. However, in some embodiments, the low-e stack 604 may only be formed above isolated portions of the transparent substrate 602. Although the layers may be described as being formed “above” the previous layer (or the substrate), it should be understood that in some embodiments, each layer is formed directly on (and adjacent to) the previously provided/formed component (e.g., layer). In some embodiments, additional layers may be included between the layers, and other processing steps may also be performed between the formation of various layers.

Still referring to FIG. 6, the protective layer 606 is formed above the transparent substrate 602. The protective layer 606 may be made of dielectric material, such as silicon nitride, and have a thickness of, for example, between about 5 nanometers (nm) and about 30 nm, such as about 10 nm. The protective layer 606 may protect the other layers in the low-e stack 604 from any elements which may otherwise diffuse from the transparent substrate 602 and may be used to tune the optical properties (e.g., transmission) of the low-e stack 604 and/or the low-e panel 600 as a whole. It should be noted that in some embodiments, the protective layer 606 is not included in the low-e stack 604.

The reflective layer 608 is formed above the protective layer 606. In some embodiments, the reflective layer 608 is made of silver and has a thickness of, for example, between about 10 nm and about 30 nm, such as about 20 nm. In some embodiments, the reflective layer 608 includes (or is made of) copper and/or gold (perhaps in addition to silver). As is commonly understood, the reflective layer 608 is used to reflect infra-red electro-magnetic radiation, thus reducing the amount of heat that may be transferred through the low-e panel 600.

In the depicted embodiment, the HEA layer 610 is formed above the reflective layer 608. The HEA layer 610 includes (or is made of) a high-entropy alloy (HEA), or HEA material. In some embodiments, the HEA material includes at least five (i.e., five or more) metallic elements in appreciable amounts by weight. For example, in some embodiments, the HEA material includes between about 5% and about 35% of each of the constituent elements by weight (e.g., the HEA material includes 20% by weight of each of five metallic elements). Exemplary elements that may be used in the HEA material include iron, zinc, zirconium, aluminum, titanium, tungsten, tantalum, hafnium, copper, boron, niobium, chromium, hafnium, and/or any combination thereof. The HEA material may be amorphous due to, for example, divergent atomic numbers of the constituent elements. The HEA layer 610 may have a thickness of, for example, between about 2 nm and about 40 nm.

In some embodiments, the HEA layer 610 may serve as a barrier layer to protect the reflective layer 608 from subsequent processing steps and to prevent any interaction of the material of the reflective layer 608 with environmental elements (e.g., water), as well as the materials of the other layers of the low-e stack 604, which may result in undesirable optical characteristics of the low-e panel 600, such as reduced reflection of infra-red electro-magnetic radiation and poor color performance. It should be noted that HEA materials may be used to serve other functions in the low-e stack 604 besides that of being a barrier layer. Further, it should be noted that in some embodiments, an HEA material may be used in a layer that is below the reflective layer 608 (i.e., between the transparent substrate 602 and the reflective layer 608).

Still referring to FIG. 6, the capping layer (or second protective layer) 612 is formed above the HEA layer 610. The capping layer 612 may be made of the same material(s) as the protective layer 606 (e.g., silicon nitride). The capping layer 612 may have a thickness of, for example, between about 5 nm and about 30 nm, such as about 20 nm. The capping layer 612 may be used to provide additional protection for the lower layers of the stack 604 and further adjust the optical properties of the low-e panel 600. It should be noted that in some embodiments the capping layer 612 may not be included in the low-e stack 104.

Although not shown, in some embodiments, the low-e stack 604 includes additional layers, such as base layers and seed layers, which may, for example, be formed between the transparent substrate 602 and the reflective layer 608 and made of metal oxides.

After the formation of the low-e stack 604, the low-e panel 600 may undergo a heat treatment to, for example, temper the glass within the transparent substrate 602. For example, the low-e panel 600 may be heated to a temperature of between about 600° C. and about 700° C. for about 30 minutes. In some embodiments, due to the nature of the HEA material, the HEA material may remain amorphous after the heat (e.g., due to divergent atomic numbers of the constituent elements).

One skilled in the art will appreciate that the embodiment(s) depicted in FIG. 6 is a “single silver” low-e panel (i.e., having one reflective/silver layer). However, in some embodiments, the low-e panel 600 (or the low-e stack 604) is formed as a “double silver,” or even a “triple silver,” low-e panel (i.e., having two or three reflective/silver layers, respectively). In such embodiments, other layers in the low-e stack 604, may be replicated along with the reflective layer, such as additional HEA layers 608.

It should also be understood that the low-e panel 600 may be a portion of (or installed in) a larger, more complex device or system, such as a low-e window. Such a window may include multiple glass substrates (or panes), other coatings (or layers), such a thermochromic coating formed on a different pane than the low-e stack, and various barrier or spacer layers formed between adjacent panes.

HEAs have various properties which may make their utilization in low-e panels desirable. HEAs have high hardness due to the lack of dislocations and immobility of the matrix atoms, as well as high resistivity to corrosion due to the lack of grain boundaries. Further, HEAs may provide improved transmission of visible light due to decreased electron mobility (i.e., relative to more conductive metals), which allows the use of thicker metal films. HEAs may also provide excellent diffusion barrier properties due to the dense packing of atoms, as well as relatively low coefficients of thermal, which allows better thermal matching to glass.

As such, the use of HEAs in low-e panels may provide improved performance with respect to emissivity, durability, and optical performance, as well as overall improve performance balance. Additionally, the use of HEAs in low-e panels may allow the total number of layers in the low-e stack to be reduced, thus simplifying the low-e stack and increasing through-put (i.e., reducing manufacturing time). The manufacturing time may further be reduced when low-Z elements are utilized, as they may be sputtered at rates higher than that of high-Z elements (e.g., tungsten, molybdenum, etc.).

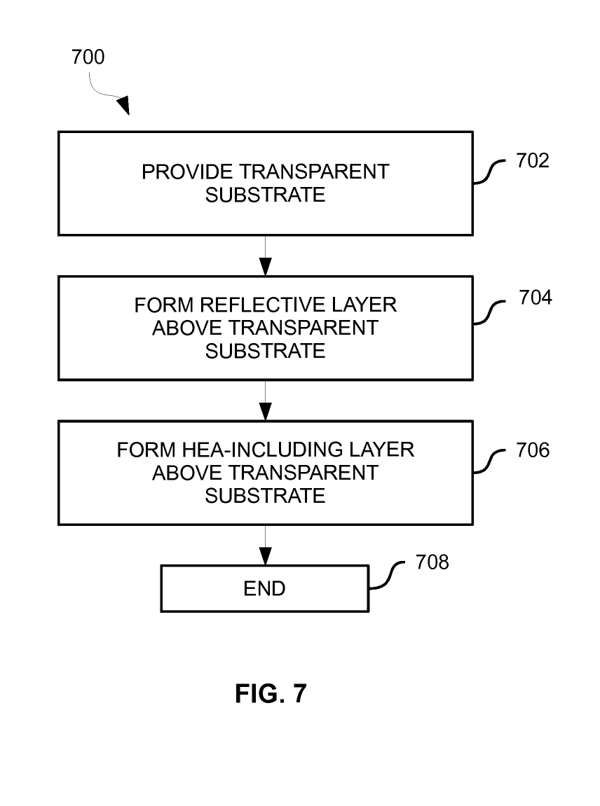

FIG. 7 is a flow chart illustrating a method 700 for forming low-e panels according to some embodiments. The method 700 begins at block 702 by providing a transparent substrate, such as the examples described above (e.g., glass).

At block 702, a reflective layer is formed above the transparent substrate. In some embodiments, the reflective layer includes (or is made of) silver.

At block 706, a layer including an HEA is formed above the transparent substrate. As described above, the HEA-including layer includes a material that is made of at least five metallic elements in appreciable amounts by weight. For example, in some embodiments, the HEA material includes between about 5% and about 35% of each of the constituent elements by weight (e.g., the HEA material includes 20% by weight of each of five metallic elements). Exemplary elements that may be used in the HEA material include iron, zinc, zirconium, aluminum, titanium, tungsten, tantalum, hafnium, copper, boron, niobium, chromium, hafnium, and any combination thereof. In some embodiments, the HEA-including layer is formed above the reflective layer, while is some embodiments, the HEA-including layer is formed between the transparent substrate and the reflective layer.

In some embodiments, the HEA-including layer is formed by positioning the transparent substrate relative to at least two targets (e.g., within the processing chamber of a PVD tool) that jointly include the metallic elements described above, and causing material to be ejected from the targets and deposited onto the transparent substrate.

Although not shown in FIG. 7, the method 700 may also include the formation of various other components/layers suitable for a low-e panel, such as (at least one) dielectric layers, seed layers, etc. (formed either above the HEA-including layer and/or between the transparent substrate and the HEA-including layer). Additionally, the method 700 may include performing a heat treatment to, for example, temper the glass within the transparent substrate. For example, the low-e panel may be heated to a temperature of between about 600° C. and about 700° C. for about 30 minutes. At block 708, the method ends.

Further, in some embodiments, the HEA-including layer (and/or other components of the low-e panels) may be formed in accordance with the principles of combinatorial processing, such as those described above (e.g., forming HEA materials above different regions on the substrate(s) using varying processing conditions, and then evaluating those materials to determine which are most suitable for particular applications).

Thus, in some embodiments, methods for forming a low-e panel are provided. A transparent substrate is provided. A reflective layer is formed above the transparent substrate. A metallic layer is formed above the transparent substrate. The metallic layer includes a high-entropy alloy.

In some embodiments, methods for evaluating low-e panel materials are provided. At least one substrate is provided. The at least one substrate has a plurality of site-isolated regions defined thereon. A first metallic material is formed above a first of the plurality of site-isolated regions with a first set of processing conditions. A second metallic material is formed above a second of the plurality of site-isolated regions with a second set of processing conditions. The first metallic material and the second metallic material are characterized. At least one of the first metallic material and the second metallic material includes a high-entropy alloy. The second set of processing conditions is different than the first set of processing conditions.

In some embodiments, methods for evaluating low-e panel materials are provided. At least one substrate is provided. The at least one substrate has a plurality of site-isolated regions defined thereon. A first high-entropy alloy material is formed above a first of a plurality of site-isolated regions with a first set of processing conditions. A second high-entropy alloy material is formed above a second of the plurality of site-isolated regions with a second set of processing conditions. The first high-entropy alloy material and the second high-entropy alloy material are characterized. The second set of processing conditions is different than the first set of processing conditions.

In some embodiments, methods for evaluating low-e panel materials are provided. At least one transparent substrate is provided. The at least one transparent substrate has a plurality of site-isolated regions defined thereon. A first high-entropy alloy material is formed above a first of the plurality of site-isolated regions with a first set of processing conditions. A second high-entropy alloy material is formed above a second of the plurality of site-isolated regions with a second set of processing conditions. The first high-entropy alloy material and the second high-entropy alloy material are characterized. Each of the first high-entropy alloy material and the second high-entropy alloy material includes between about 5% and about 35% of each of at least five metallic elements by weight. The second set of processing conditions is different than the first set of processing conditions.

In some embodiments, low-e panels are provided. The low-e panels include a transparent substrate. A reflective layer is formed above the transparent substrate. A metallic layer is formed above the transparent substrate. The metallic layer includes a high-entropy alloy.

Although the foregoing examples have been described in some detail for purposes of clarity of understanding, the invention is not limited to the details provided. There are many alternative ways of implementing the invention. The disclosed examples are illustrative and not restrictive.

What is claimed:

1. A method for evaluating materials, the method comprising:
providing a substrate, wherein the substrate has a plurality of site-isolated regions defined thereon;
forming a first metallic material above a first of the plurality of site-isolated regions with a first set of processing conditions;
forming a second metallic material above a second of the plurality of site-isolated regions with a second set of processing conditions; and
characterizing the first metallic material and the second metallic material,
wherein at least one of the first metallic material or the second metallic material comprises a high-entropy alloy, and wherein the second set of processing conditions is different than the first set of processing conditions.

2. The method of claim 2, wherein the high-entropy alloy comprises at least five metallic elements.

3. The method of claim 2, wherein the high-entropy alloy comprises between about 5% and about 35% of each of the at least five metallic elements by weight.

4. The method of claim 3, wherein the at least five metallic elements comprises five or more of iron, zinc, zirconium, aluminum, titanium, tungsten, tantalum, hafnium, copper, boron, niobium, chromium, hafnium, or a combination thereof.

5. The method of claim 3, wherein the forming of each of the first metallic material and the second metallic material each comprises positioning the at least one substrate relative to at least two targets.

6. The method of claim 5, wherein the forming of each of the first metallic material and the second metallic material each comprises causing material to be ejected from the at least two targets.

7. The method of claim 6, further comprising forming at least one dielectric layer above the at least one substrate.

8. The method of claim 7, further comprising forming a reflective layer above the at least one substrate.

9. The method of claim 1, wherein the characterizing the first metallic material and the second metallic material is performed using ellipsometry, atomic force microscopy (AFM), scanning electron microscopy (SEM), optical transmission and reflectance testing, X-Ray Diffraction (XRD), X-Ray Fluorescence (XRF), Fourier Transform Infrared (FTIR) spectroscopy, or a combination thereof.

10. A method for evaluating materials, the method comprising:
providing a substrate, wherein the substrate has a plurality of site-isolated regions defined thereon;
forming a first high-entropy alloy material on a first of the plurality of site-isolated regions with a first set of processing conditions;
forming a second high-entropy alloy material on a second of the plurality of site-isolated regions with a second set of processing conditions; and
characterizing the first high-entropy alloy material and the second high-entropy alloy material,
wherein the second set of processing conditions is different than the first set of processing conditions.

11. The method of claim 10, the characterizing the first high-entropy alloy material and the second high-entropy alloy material is performed using ellipsometry, atomic force microscopy (AFM), scanning electron microscopy (SEM), optical transmission and reflectance testing, X-Ray Diffraction (XRD), X-Ray Fluorescence (XRF), Fourier Transform Infrared (FTIR) spectroscopy, or a combination thereof, and further comprising selecting one of the first set of processing conditions or the second set of processing conditions based on the characterizing of the first high-entropy alloy material and the second high-entropy alloy material.

12. The method of claim 10, wherein the first high-entropy alloy material and the second high-entropy alloy material each comprise between about 5% and about 35% of each of at least five metallic elements by weight.

13. The method of claim 12, wherein the at least five metallic elements comprises five or more of iron, zinc, zirconium, aluminum, titanium, tungsten, tantalum, hafnium, copper, boron, niobium, chromium, hafnium, or a combination thereof.

14. The method of claim 10, wherein the forming of the first high-entropy alloy material and the forming of the second high-entropy alloy material occur simultaneously.

15. A method for evaluating materials, the method comprising:
providing a transparent substrate, wherein the transparent substrate has a plurality of site-isolated regions defined thereon;
forming a first high-entropy alloy material above a first of the plurality of site-isolated regions with a first set of processing conditions;
forming a second high-entropy alloy material above a second of the plurality of site-isolated regions with a second set of processing conditions; and
characterizing the first high-entropy alloy material and the second high-entropy alloy material,
wherein each of the first high-entropy alloy material and the second high-entropy alloy material comprises between about 5% and about 35% of each of at least five metallic elements by weight, and wherein the second set of processing conditions is different than the first set of processing conditions.

16. The method of claim 15, wherein the at least five metallic elements comprises five or more of iron, zinc, zirconium, aluminum, titanium, tungsten, tantalum, hafnium, copper, boron, niobium, chromium, hafnium, or a combination thereof.

17. The method of claim 16, wherein the forming of each of the first high-entropy alloy material and the second high-entropy alloy material comprises positioning the at least one transparent substrate relative to at least two targets.

18. The method of claim 17, wherein the forming of each of the first high-entropy alloy material and the second high-entropy alloy material comprises causing material to be ejected from the at least two targets.

19. The method of claim 18, wherein the characterizing the first high-entropy alloy material and the second high-entropy alloy material is performed using ellipsometry, atomic force microscopy (AFM), scanning electron microscopy (SEM), optical transmission and reflectance testing, X-Ray Diffraction (XRD), X-Ray Fluorescence (XRF), Fourier Transform Infrared (FTIR) spectroscopy, or a combination thereof, and further comprising selecting one of the first set of processing conditions or the second set of processing conditions based on the characterizing of the first high-entropy alloy material and the second high-entropy alloy material.
